(19) United States
(12) Patent Application Publication
(10) Pub. No.: US 2015/0024656 A1
(43) Pub. Date: Jan. 22, 2015

(54) Hands Free Breast Shield Cover

(71) Applicant: Amy Beth Johnson, Collierville, TN (US)

(72) Inventor: Amy Beth Johnson, Collierville, TN (US)

(21) Appl. No.: 13/943,978

(22) Filed: Jul. 17, 2013

(51) Int. Cl. A41C 3/04; A61M 1/06

(52) U.S. Cl. CPC A41C 3/04; A61M 1/066
USPC 450/36

(57) ABSTRACT

A breast shield cover designed to hold a breast shield in place during breast pumping without the wearer having to hold the breast shield in place.

### CROSS-REFERENCE TO RELATED APPLICATIONS

| | | |
|---|---|---|
| 6,855,029 | February, 2005 | Rothman |
| 7,076,809 | July, 2006 | Rothman |
| 4,633,876 | November, 1984 | Scullin |
| 4,911,677 | January, 1989 | White |
| 5,094,647 | March, 1991 | Courtney |
| 5,697,830 | August, 1996 | White |
| 6,319,092 | September, 2000 | Leyhe and Warner |
| 6,361,398 | February, 2001 | Knapp |
| 8,147,291 | April, 2009 | Kirtz |
| 8,118,638 | September, 2009 | Forbes |
| 2012122,040 | May, 2008 | Grayson |
| 2012893,911 | September, 2010 | Drew |

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC

Not Applicable

### DESCRIPTION

1. Field of the Invention

This invention relates to the allowing the wearer of this disclosure to perform hands free breast pumping when the breast shield cover is affixed to a nursing tank top or nursing brassiere.

2. Background of the Invention

For nursing mothers who supplement breast feeding with breast pumping, so that breast milk can be stored for future use, breast pumping breast pumping is a time consuming process that limits the mother's ability to perform other tasks during the procedure. In the present disclosure, a breast shield cover is affixed to the internal portion of a nursing tank top or nursing brassiere. The breast shield cover is designed with two holes where the wearer's nipple region would be when affixed to the nursing tank top or nursing brassiere. These holes allow a breast shield to protrude through the holes without passing entirely through. When the beast shield cover is affixed to the nursing tank top or nursing brassiere, the breast shield cover is pulled tight enough for the breast shield that is protruding through the breast shield cover to form a seal over the nipple region without hand support, thus allowing hands free breast pumping.

In U.S. Pat. No. 6,855,029, Rothman discloses a stretchable nursing tank top with a cover that can be folded down to expose the wearer's breast. In the present disclosure, additional fasteners would be attached to the stretchable nursing tank top to allow the breast shield cover to be attached and allow the wearer to perform hands free breast pumping.

In U.S. Pat. No. 7,076,809, Rothman discloses a stretchable nursing tank top with a cover that can be folded down to expose the wearer's breast. In the present disclosure, additional fasteners would be attached to the stretchable nursing tank top to allow the breast shield cover to be attached and allow the wearer to perform hands free breast pumping.

In U.S. Pat. No. 4,633,876, Scullin discloses a brassiere for use in nursing and maternity, as well as for use as a prosthetic device. Such use requires cups which can be opened and closed easily without having to unfasten the brassiere from the wearer, but does not disclose a nursing brassiere that allows the attachment of a breast shield cover.

In U.S. Pat. No. 4,911,677, White discloses a nursing brassiere having a frontal section comprised of partially overlapping frontal portions each adapted to cover one side of a wearer's chest area, but does not disclose a nursing brassiere that will allow the attachment of a breast shield cover.

In U.S. Pat. No. 5,094,647, Courtney discloses a nursing brassiere that includes a cup which fits over a breast of the user, the cup includes a flap for selectively concealing or exposing the breast but does not disclose a nursing brassiere that will allow the attachment of a breast shield cover.

In U.S. Pat. No. 5,697,830, White discloses a nursing brassiere with overlapping frontal portions that provides added support and yet can be easily pulled aside to provide nursing access but does not disclose a nursing brassiere that will allow the attachment of a breast shield cover.

In U.S. Pat. No. 6,319,092, Leyhe and Warner disclose a nursing brassiere that allows the mother to change out only a wet section of the brassiere without having to remove clothing and replace the entire brassiere but does not disclose a nursing brassiere that will allow the attachment of a breast shield cover.

In U.S. Pat. No. 6,361,398, Knapp discloses a brassiere or garment with dual exterior brassiere cup covers released and re-fastened beneath the underarms and configured to function at least in part in the manner of a brassiere and usable by a nursing mother for improved ease of nursing an infant child but does not disclose a nursing brassiere that will allow the attachment of a breast shield cover.

In U.S. Pat. No. 8,147,291, Hirtz discloses a convertible nursing brassiere having brassiere support cups releasable from straps to provide access and further including underlying support panels connected to the encircling strap or under wire support and remaining connected to the straps to provide continued support during nursing. The release is located either under the arm or at the connection with the shoulder strap. However, U.S. Pat. No. 8,147,291 does not disclose a nursing brassiere that will allow the attachment of a breast shield cover.

In U.S. Pat. No. 8,118,638, Forbes discloses a nursing brassiere having a primary band assembly for securing said nursing brassiere to a user's upper chest and a separate secondary band that allows slidable movement of each of the breast cups for uncovering a breast during a nursing application but does not disclose a nursing brassiere that will allow the attachment of a breast shield cover.

In U.S. Patent Appl. 2012122,040, Grayson discloses a nursing brassiere providing nipple protection against discomfort associated with breast-feeding having two identical cups, each having a centered aperture to expose a woman's nipple and interchangeable pad and shield attached via snaps to cover the aperture and a flap to cover the pad or shield. The pad is flexible and absorbent to prevent leaking, while the shield has a nipple with a drinking opening to allow an infant to breast feed. The shield also has a plastic ring seal to prevent leaking onto a woman's skin or clothes during breast feeding. However, 2012122,040 does not disclose a nursing brassiere that will allow the attachment of a breast shield cover.

In U.S. Patent App. 2012893,911, Drew discloses a brassiere comprising breast supporting cups with a frontal opening to expose the breast, the breast-supporting cup comprising a panel/flap that covers the opening and a part of which is releasably attachable to the shoulder strap of the brassiere. The clasp on the shoulder strap is movable up and down the shoulder strap to a selected position and having a clamping part so that it may be clamped at the selected position, thereby providing adjustment of the breast-supporting cup. However, 2012893,911 does not disclose a nursing brassiere that will allow the attachment of a breast shield cover.

### SUMMARY OF THE INVENTION

A breast shield cover that when affixed to a nursing tank top or nursing brassiere stretches tight enough to hold a breast shield over the wearer's nipple regions, where said breast shield can create a seal around the nipple and allow for hands free breast pumping.

### BRIEF DESCRIPTION OF THE SEVERAL VIEWS OF THE DRAWING

FIG. 1 is a display of a representative embodiment of the breast shield cover with attachments and two holes;

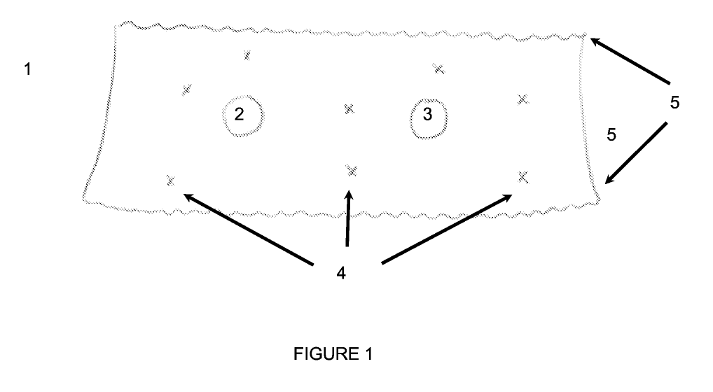

FIG. 2 is a display of a representative embodiment of the nursing tank top with the cover folded down, exposing where the attachments are located so that the breast shield cover can be attached;

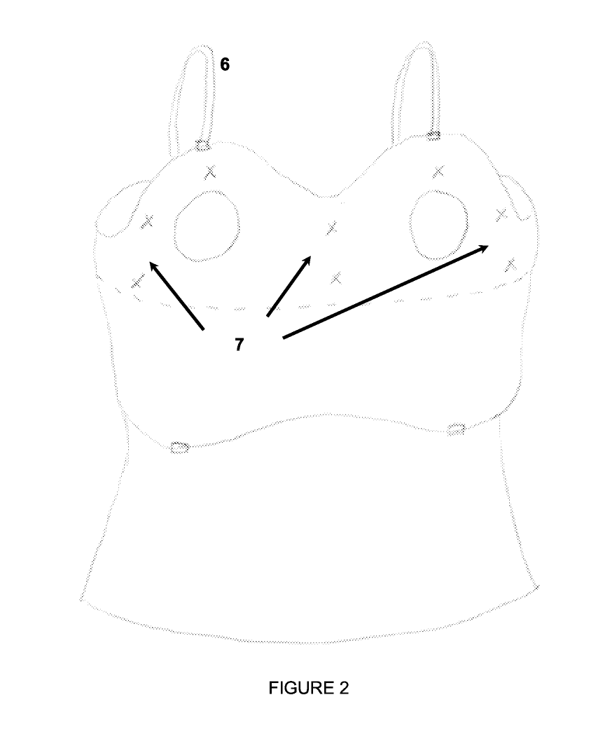

FIG. 3 is a representative embodiment of the stretchable nursing brassiere with the breast covers folded down, exposing where the attachments are located so that the breast shield cover can be attached;

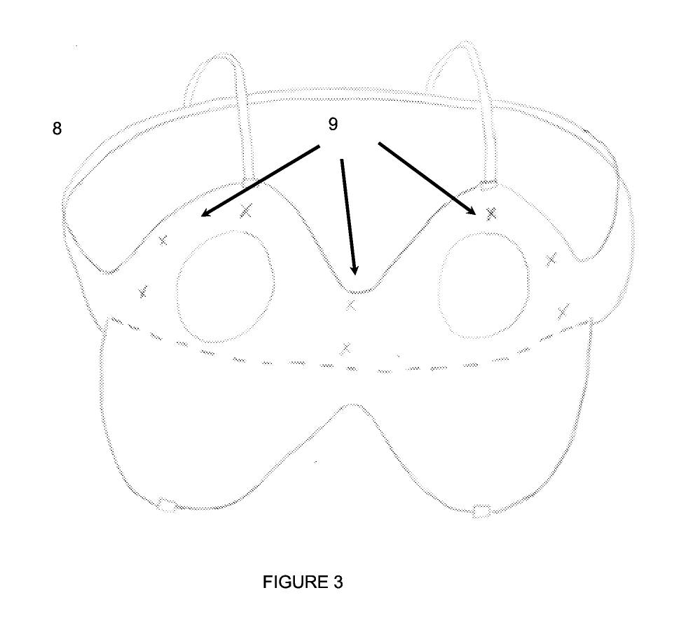

FIG. 4 is a representative embodiment of the display of the nursing brassiere with a breast shield cover attached, with a breast shield in place;

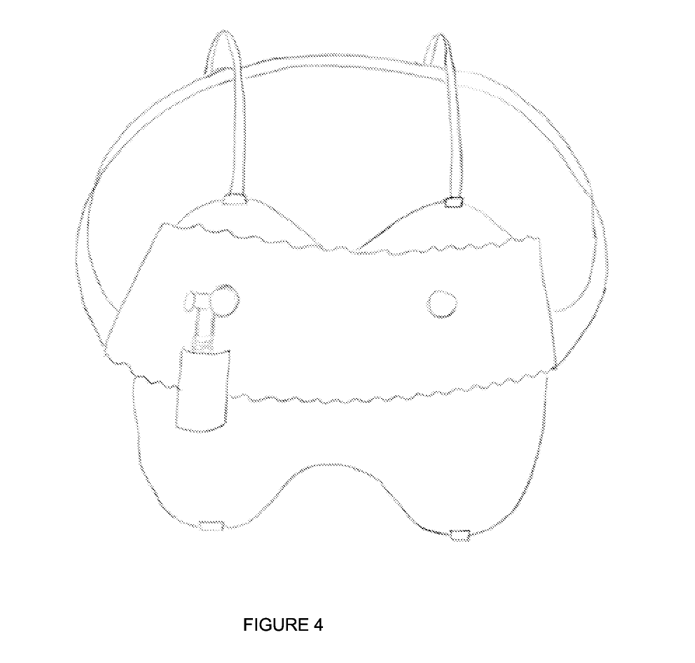

### DETAILED DESCRIPTION OF THE INVENTION

A breast shield cover 1 made of fabric, including two holes 2 and 3 located where the wearer's nipples would protrude when wearing, a plurality of fasteners 4 to affix the breast shield cover to a nursing tank top, nursing brassiere or similar garment, with appropriate elasticity in the fabric or by means of elastic banding at the upper 5 or lower edges of the breast shield cover.

In one preferred embodiment the breast shield cover can be affixed to a nursing tank top 6 by means of a plurality of fasteners located on the nursing tank top in such a way that the plurality of fasteners on the breast shield cover match up to the plurality of fasteners on the nursing tank top so that the breast shield cover can be affixed to the nursing tank top. When attached to the nursing tank top, the breast shield cover stretches over the breasts of the wearer to hold breast shields in place against the wearer's nipple region tight enough to form seals. The breast shield can then protrude through the holes of the breast shield cover, be attached to a breast pumping apparatus, and milk extraction can be facilitated without the need for holding the breast shield in place.

In a second preferred embodiment the breast shield cover can be affixed to a nursing brassiere 7 by means of a plurality of fasteners 8 located on the nursing brassiere in such a way that the plurality of fasteners on the breast shield cover match up to the plurality of fasteners on the nursing brassiere so that the breast shield cover can be affixed to the nursing brassiere. When affixed to the nursing brassiere, the breast shield cover stretches over the breasts of the wearer to hold breast shields in place against the wearer's nipple region tight enough to form seals. The breast shield can then protrude through the holes of the breast shield cover, be attached to a breast pumping apparatus, and milk extraction can be facilitated without the need for holding the breast shield in place.

What is claimed:

1. A breast shield cover comprising fabric with two holes in the fabric located in the wearer's nipple regions when worn;
a plurality of fasteners to affix the fabric to the wearer's undergarment;
where, when the fabric is affixed to the wearer's undergarment and the wearer has a breast shield cover over her right nipple, the fabric stretches to hold the breast shield in place without the aid of the wearer, creating a seal between the breast shield and the nipple region such that milk extraction can take place during breast pumping, leaving the wearer's hands free during the process; and
where, when the fabric is affixed to the wearer's undergarment and the wearer has a breast shield cover over her left nipple, the fabric stretches to hold the breast shield in place without the aid of the wearer, creating a seal between the breast shield and the nipple region such that milk extraction can take place during breast pumping, leaving the wearer's hands free during the process.

2. The breast shield cover of claim 1 where the undergarment is a nursing tank top with a plurality of fasteners located so that the fasteners on the breast shield cover and the fasteners on the nursing tank top match up and can be affixed so that the breast shield cover is held in place as described.

3. The breast shield cover of claim 2 where the edges of the holes in the fabric are reinforced.

4. The breast shield cover of claim 1 where the undergarment is a nursing brassiere with a plurality of fasteners located so that the fasteners on the breast shield cover and the fasteners on the nursing brassiere match up and can be affixed so that the breast shield cover is held in place as described.

5. The breast shield cover of claim 4 where the edges of the holes in the fabric are reinforced.
